(19) United States
(12) Patent Application Publication
(10) Pub. No.: US 2026/0354714 A1
(43) Pub. Date: Nov. 12, 2026

(54) COMPUTER-IMPLEMENTED METHOD OF MONITORING A TRANSCUTANEOUS SENSOR FOR MEASURING PARTIAL PRESSURE OF ONE OR MORE BLOOD GASES OF A PATIENT

(71) Applicant: Radiometer Medical ApS, Brønshøj (DK)

(72) Inventors: Niels Erik OLESEN, Brønshøj (DK); Søren Jacob BRUN, Sønshøj (DK)

(21) Appl. No.: 19/130,772

(22) PCT Filed: Dec. 21, 2023
(86) PCT No.: PCT/EP2023/087344

§ 371 Date: May 16, 2025

(30) Foreign Application Priority Data
Dec. 22, 2022 (EP) 22216197.8

(51) Int. Cl. A61B 5/145; A61B 5/1477; A61B 5/1495

(52) U.S. Cl. CPC A61B 5/14542; A61B 5/1477; A61B 5/1495

(57) ABSTRACT

According to a first aspect of the present invention, a computer-implemented method of monitoring a transcutaneous sensor configured for measuring partial pressure of one or more blood gases of a patient is presented. The method comprises the steps of determining a response time parameter of the transcutaneous sensor (step S1). The method further comprises the step of determining, based on the determined response time parameter, whether a partial pressure measuring capability of the sensor is sufficient (step S2). This method may not only detect an increase of the response time of the sensor due to a dry membrane, which may occur after several days of using a membrane with the sensor. The method may also detect deterioration of the sensor, which may occur only after several years of using the sensor.

### FIELD OF THE INVENTION

The present invention relates to a computer-implemented method of monitoring a transcutaneous sensor configured for measuring partial pressure of one or more blood gases of a patient, to a computer program, a non-transitory program storage medium storing such a program, a transcutaneous blood gas monitor for transcutaneously monitoring one or more blood gases of a patient, and to the use of a result of a determination of a response time parameter of a transcutaneous sensor.

### TECHNICAL BACKGROUND

Transcutaneous monitoring is the method to use in patients who have a need for continuous and non-invasive monitoring of oxygenation and ventilation. Through a sensor applied to the body, blood gases diffusing through the skin can be detected and measured. In detail, transcutaneous monitoring is a useful and widely used trending tool to monitor oxygenation (tcpO₂) and ventilation (tcpCO₂) status non-invasively of patients. It provides vital real-time information, allowing one to take immediate action when needed to improve patient safety and comfort. One exemplary transcutaneous monitor of Radiometer Medical ApS is the TCM5 FLEX transcutaneous monitor, which is a compact, easy-to-use solution that delivers accurate and continuous measurement of oxygenation (tcpO₂) and ventilation (tcpCO₂) status in neonatal, pediatric and adult patients in the intensive care units (ICU).

Such a transcutaneous monitor typically comprises a transcutaneous electrochemical sensor configured for measuring partial pressure blood gases of the patient. Such sensors need from time to time a re-membraning, i.e., the replacement of the membrane used within said sensor. Currently, the re-membraning interval of the sensors is typically fixed to a certain time period, e.g., 28 days, as the risk of the sensor membrane drying out is significantly increased or too high after this 28-days period. When the sensor membrane dries out, the measurement will become incorrect.

During research and development, the inventors of the present invention have identified the need to extend the re-membraning interval beyond the current fixed time period. They have also identified the need to detect a sensor that for some reason has dried out before the fixed time period has elapsed and to also detect a sensor where the performance has decreased over time and due to use such that the response time is too high.

It is, therefore, desirable to provide for an improved monitoring of a transcutaneous sensor configured for measuring partial pressure of one or more blood gases of a patient. This is achieved with the present invention as will be explained in more detail hereinafter.

Aspects of the present invention, embodiments, examples and exemplary steps are disclosed in the following. Different embodiments, examples and exemplary features of the invention can be combined in accordance with the invention wherever technically expedient and feasible.

### SUMMARY OF THE INVENTION

As stated above, it may be desirable to provide for an improved monitoring of a transcutaneous sensor configured for measuring partial pressure of one or more blood gases of a patient.

This is achieved by the subject-matter of the independent claims, wherein further embodiments are incorporated in the dependent claims and the following description and figures.

Technical terms are used by their common sense. If a specific meaning is conveyed to certain terms, definitions of terms will be given in the following in the context of which the terms are used.

According to a first aspect of the present invention, a computer-implemented method of monitoring a transcutaneous sensor configured for measuring partial pressure of one or more blood gases of a patient is presented. The method comprises the steps of determining a response time parameter of the transcutaneous sensor (step S1). The method further comprises the step of determining, based on the determined response time parameter, whether a partial pressure measuring capability of the sensor is sufficient (step S2).

As will become apparent from the present disclosure, the suggested method uses the results of the response time determination during the monitoring of the sensor. The method requires that a response time parameter is determined in step S1, the results of which are then used for the determination whether the capability of the sensor for measuring the partial pressure of the blood gases is sufficient, i.e., adequate and/or acceptable. Different possibilities like e.g., the comparison of a measured response time of an individual sensor with a response time threshold are disclosed for determining whether the capability of the sensor is sufficient or not. Therefore, the step S2 of the presented method may be seen as an evaluation of the partial pressure measuring capability of the sensor. In particular, an evaluation of the response time parameter is carried out for deciding whether the capability of the sensor for measuring partial gas pressure is sufficient, adequate and/or acceptable/accepted. As will be appreciated by the skilled reader, in the context of the present invention, the term “sufficient” shall be used synonymously with the term “adequate” and “acceptable”. Different possibilities like e.g., the comparison of a measured response time with a response time threshold are disclosed hereinafter for determining whether the capability of the sensor is sufficient or not. Thus, in general, the method determines in step S2 whether the determined response time parameter fulfils or meets a sufficiency criterion, i.e., an acceptance criterion. This sufficiency criterion, i.e., an acceptance criterion, may preferably be predefined. Thus, as is understood by the skilled reader, in step S2 it is determined, based on the determined response time parameter, whether the partial pressure measuring capability of the sensor is considered—by the computer implemented method—as acceptable/accepted. Therefore, step S2 shall be understood as determining, based on the determined response time parameter, whether a partial pressure measuring capability of the sensor is sufficient thereby determining whether the determined response time parameter fulfils or meets a sufficiency criterion, i.e., an acceptance criterion.

As will be detailed in particular embodiments hereinafter, the determination whether said capability of the sensor is sufficient comprises or may be embodied as determining whether a re-membraning of the sensor, or a replacement of the sensor, or a maintenance of the sensor is to be initiated. In other words, in said embodiments the method determines in step S2, based on the response time parameter determined in step S1, whether a re-membraning, a replacement of the sensor or a maintenance of the sensor is needed. This will be explained in more detail in the context of particular embodiments hereinafter.

As will be explained in detail hereinafter, the presented method may not only detect an increase of the response time of the sensor due to a dry membrane, which may occur after several days of using a membrane with the sensor. The method may also detect deterioration of the sensor, which may occur only after several years of using the sensor. It could be less than several years depending on the use and settings of the sensor. For example, the surface of the glass of the sensor may not be adequate anymore after several years. Therefore, besides the measure of re-membraning the sensor, the sensor may also be repaired or refurbished by using e.g., hydrofluoric acid on the glass to restore the glass surface. Therefore, besides detecting the need for re-membraning, the presented method may also detect the need for replacement or maintenance of the sensor. As mentioned before, this also covers repairing, refurbishing, servicing or re-activating the glass surface of the transcutaneous sensor. In general, the method may detect any kind of sensor condition that disturbs or changes the capability of the sensor to measure the partial pressure of the blood gas, which capability change is detectable based on evaluating in step S2 the response time parameter determined in step S1.

Note that different response time parameters may be used for the determination carried out in step S1. For example, said response time parameter determined in step S1 can be a value, in particular an absolute value, of the response time. Note that in the context of the present invention and if not stated differently, the response time shall be understood as the so called 10% to 90% response time, i.e., the time which is needed by the sensor to detect a change from a first partial gas pressure to a second partial gas pressure, where 10% and 90% refer to the time difference between detecting 10% and 90% of the difference between the first and second partial gas pressures. This can also be related to the time constant T described herein, see e.g., the description of FIG. 4. The response time is not necessarily the 10% to 90% response time, but could in other embodiments be, e.g., the 20% to 80% response time, the 5% to 95% response time or any other percentages chosen on the response curve. The person skilled in this art is aware of the parameter of the (10% to 90% or other) response time. Further, the skilled reader is aware that the (10% to 90% or other) response time can be converted by calculation into the time constant T which is known by the skilled reader as the time constant of an exponential or bi-exponential response of the transcutaneous sensor in response to a suddenly changing gas pressure. Thus, in line with the understanding of the skilled reader, the response time as used herein preferably refers to said x % to y % response time, e.g., 10% to 90% response time or to the time constant T. This will be explained in more detail hereinafter.

Moreover, said response time parameter determined in step S1 may also be embodied as a trend parameter, which is indicative of a time development of the response time of the transcutaneous sensor. A non-limiting example of such a trend parameter is a time derivative of a measurement curve of said blood gas partial pressure values, which are measured over time by the monitored sensor. This can be gathered from and will be elucidated further in context of FIGS. 2 and 3. Moreover, other examples of response time parameters to be determined in step S1 can be used.

Moreover, the method step S1 “determining a response time parameter” may be embodied as calculating a response time parameter and/or as measuring a response time parameter. This will be elucidated hereinafter in the context of particular embodiments. Note that the step S1 of determining the response time parameter covers any calculation using a two-point model, and it also covers for example the use of regression analysis, as will be explained in more detail hereinafter. In another embodiment, this determination of step S1 is embodied as a prediction of the response time parameter by using a predictive maintenance module, e.g., using an artificial intelligence module and/or machine learning.

The presented method may be carried out by, for example, a transcutaneous blood gas monitor and can be repeated regularly, for example every 24 hours, every 12 hours, every 8 hours, or the like. Thus, the method of monitoring a transcutaneous sensor may be seen as a method of making continuous quality checks of the transcutaneous sensor. Moreover, the presented method of monitoring a transcutaneous sensor may be seen as a method of calibrating the transcutaneous sensor or may be part of a calibration method which calibrates said transcutaneous sensor.

Furthermore, in the context of the present invention, the term “transcutaneous sensor” shall be understood as any kind of sensor which is configured for non-invasively measuring in/on the skin of a patient a partial pressure of one or more blood gases of a human being.

It should be noted that the presented method may be used for several different kinds of transcutaneous sensors. In particular, the presented method may be used with electrochemical transcutaneous sensors for measuring partial pressure of one or more blood gases of a patient, particularly for electrochemical transcutaneous STOW-SEVERINGHAUS TYPE CO₂ sensors, amperometric O₂ sensors, as well as other sensors. Moreover, the presented method can also be applied to transcutaneous sensors using optical sensor technology. The working principle of an optical sensor may be different from an electrochemical sensor as described here. However, response time may still be useful for determining some conditions of the sensor.

As is understood by the skilled reader, the response times should in general be measured at the same temperature to be comparable. Alternatively, the response times for different temperatures could be converted to a standard temperature through a known conversion function and thereby be made comparable. In other words, a prerequisite for making a meaningful comparison is that the response time is measured at the same temperature, e.g. set by the user to 42 °C, or that the response times for different temperatures are converted to make them comparable. This temperature is normally set for the entire period of measuring. The monitor can be set to keep this temperature between uses or go into standby when not used where the temperature is lowered.

As will be appreciated by the skilled reader, by implementing a response time parameter determination in a monitoring algorithm as suggested herein, a notification about re-membraning, sensor replacement or sensor maintenance can be based on the actual sensor condition. This will typically enable a re-membraning interval which is considerably longer than for example the fixed days, e.g., 28 days, used in the prior art.

This novel method of monitoring a transcutaneous sensor leads to particular advantages. First, the re-membraning interval can be extended beyond the prior art fixed time period. Currently, re-membraning is one of the biggest disadvantages for customers using transcutaneous monitoring. Moreover, with the presented method, a sensor can be detected that for some reason has dried out before the fixed time period has elapsed. Furthermore, a sensor can be detected whether performance has decreased over time and due to use such that the response time of the sensor is too high.

As has been explained before, the current re-membraning interval of 28 days is a number determined by a safety margin. Equivalent or better safety can be provided by regularly or always monitoring the response time of the sensors, as suggested herein. This is in particular important for CO₂ sensors, as shown in and explained in the context of FIG. 2. For CO₂ sensors, the response time closely correlates with the onset of incorrect measurement values at steady state. The data in FIG. 3 show that the behaviour of the sensor suddenly changes such that the partial blood gas measurements of the sensor become less accurate. This occurs, for example, when the sensor membrane dries out and is correlated with a change in response time. This correlation is explained by the fact that the electrolyte applied to the inner membrane of the transcutaneous sensor slowly evaporates resulting in a more concentrated electrolyte. The phenomena with an increased response time on the concentration of the electrolyte is used in a particular embodiment of the present invention. Using this correlation and/or phenomena for detecting a dry transcutaneous sensor thus provides for an improved monitoring of the sensor.

Note that the method presented herein is applicable to sensors calibrated on both a single gas concentration and on two different gas concentrations.

According to another exemplary embodiment of the present invention, the response time parameter is indicative of the response time of the sensor in measuring a partial pressure of the one or more blood gases of the patient.

According to another exemplary embodiment of the present invention, the step S2 of determining whether the partial pressure measuring capability of the sensor is sufficient comprises determining whether a re-membraning of the transcutaneous sensor is to be initiated. This determination of the re-membraning is based on the previously determined response time parameter.

Particular transcutaneous sensors for blood gas measurements comprise a membrane. In particular, transcutaneous CO₂ sensors are electrochemical sensors where the membrane is provided in an electrolyte, which might evaporate. This evaporation might change the properties of the membrane and if one does not add additional electrolyte, a process of drying out of the membrane may start. Such a process, however, may cause a change of the sensor's capability of measuring the partial pressure of patients' blood gases. Therefore, with the presented embodiment, one can extend the re-membraning interval beyond the currently used fixed time period used in the prior art, and one may also detect sensors that for some reasons have dried out before the currently used fixed time period. Preferably, in step S2 it is determined, based on the determined response time parameter, whether a partial pressure measuring capability of the sensor is sufficient thereby determining whether the determined response time parameter fulfils or meets a sufficiency criterion, i.e., an acceptance criterion.

According to another exemplary embodiment of the present invention, the step S2 of determining whether the partial pressure measuring capability of the sensor is sufficient comprises the determination whether a replacement or maintenance of the transcutaneous sensor due to deterioration is to be initiated. Moreover, said determination of replacement or maintenance is based on the determined response time parameter.

In particular embodiments, the transcutaneous sensor can be repaired or refurbished by using, for example, hydrofluoric acid on the glass to restore the glass surface of the sensor. Other repairing or refurbishing measures may be used as well. Therefore, after determining that a replacement or a maintenance of the sensor is needed, a signal or alert may be generated for the user or the monitor carrying out the presented method that a replacement, repair, a refurbish, or a service of the sensor, or a reactivation of the glass surface of the sensor is needed. Further details about such signals will be explained in the context of further embodiments hereinafter. Preferably, in step S2 it is determined, based on the determined response time parameter, whether a partial pressure measuring capability of the sensor is sufficient thereby determining whether the determined response time parameter fulfils or meets a sufficiency criterion, i.e., an acceptance criterion.

According to another exemplary embodiment of the present invention, the response time parameter determined in step S1 is a value of the response time and the method comprises the step of comparing the determined value of the response time with a response time threshold.

As mentioned before, the value of the response time may be a value of the 10% to 90% response time or a value of the time constant T of the underlying exponential or bi-exponential response of the transcutaneous sensor, see e.g., FIG. 4 and the explanation thereof. As is understood by the skilled reader, particular absolute values of the response time are measured, determined, and/or used in the context of the present invention, in contrast to relative values. In other words, this embodiment details that values of the response time are measured and/or calculated and are compared with said response time threshold. The outcome of this comparison then defines whether the monitoring method concludes that a re-membraning, a replacement and/or a maintenance of the sensor is needed or not.

Note that the response time threshold as used herein can be embodied in different ways. For example, the response time threshold can depend on the initial response time of the sensor. This means that the measured response time right after re-membraning the sensor is used to set or define this response time threshold, e.g., double or triple the initial response time, or multiplying it by any other factor set by the user or the system. Thus, the individual response time of a particular sensor may be measured and/or calculated and the threshold may be set accordingly. In other words, the threshold may be individually set for an individual sensor, e.g., to the value of two times the response time that was measured after re-membraning. In another example, the response time threshold may be calculated or determined by adding a predetermined amount of time to the response time measured after re-membraning. Moreover, in particular embodiments, one may use a predetermined threshold. For example, the response time threshold may be set to a particular predetermined time period, for example to 30 s, 40 s, 50 s, or 60 s. As will be appreciated by the skilled reader, many different ways of using predefined or individually set thresholds can be used in the context of the present invention.

According to another exemplary embodiment, the method comprises the step of generating a control signal configured for causing a re-membraning of the sensor or configured for causing a replacement or maintenance of the sensor if the determined value of the response time exceeds the response time threshold.

Such a control signal allows the device carrying out this monitoring method to deactivate the sensor functionality and/or to alert the user that a re-membraning, replacement and/or maintenance is needed. The control signal will thus be generated when it was determined or decided that a re-membraning is necessary, i.e., when the determined response time of the sensor exceeds, i.e., is longer, than the response time threshold. In an example, the response time of a sensor is 54 s and the response time threshold is 50 s. In this case, a re-membraning is needed and a corresponding signal or alert is generated.

According to another exemplary embodiment of the present invention, the response time parameter determined in step S1 is a trend parameter indicative of a time development of the response time of the transcutaneous sensor. The method further comprises the step of comparing the determined trend parameter with a trend threshold regarding the time development of the response time of the transcutaneous sensor (step S3b).

As will be explained in detail in the context of particular embodiments, one may use for example for the trend parameter a first and/or a second time derivative of a measurement curve as shown in any of FIGS. 2 to 4. Moreover, the trend threshold used in step S3b in preferred embodiments is a set threshold value of the first or second time derivative of said measurement curves shown in FIGS. 2 to 4.

FIG. 2 shows the partial CO₂ pressure values measured by a monitored sensor when being exposed to a significant change in gas pressure. For example, if the steepness, i.e., the first time derivative, of a partial blood gas pressure measured by the sensor over time is too low in the first part of the curve where the pressure increases, see FIG. 2, this may be used as a criterion to cause said control signal. Said first part of the curve may be in the time period between 50 s and 300 s. As can be gathered from for example FIG. 2, the steepness of the partial blood gas pressure curve over time decreases in said first part of the curve from day 10 to day 50 of the use of the sensor.

In a further exemplary embodiment of the present invention, the control signal is configured for preventing a further use of the sensor by an operator by for example deactivating the sensor and/or the control signal is configured for alerting an operator about the need for re-membraning, a replacement or maintenance of the sensor.

Therefore, the device carrying out the method described herein, for example a transcutaneous blood gas monitor, may generate a control signal for sensor deactivation such that the user cannot use anymore the transcutaneous sensor for blood gas measurements. Moreover, the operator or user may be alerted with a visual or audio signal (e.g., via a user interface of the transcutaneous blood gas monitor) that a re-membraning of the sensor is needed, or that a replacement of the sensor is needed or that maintenance of the sensor is needed.

According to an exemplary embodiment of the present invention, the method comprises the step of generating a control signal configured for causing a re-membraning of the sensor or configured for a replacement or maintenance of the sensor if the determined trend parameter deviates from the trend parameter by at least a predetermined minimum amount (S4b).

Such a control signal may cause an automatic re-membraning of the sensor, or an automatic replacement of the sensor, or an automatic maintenance of the sensor, such that the user is not bothered with these tasks.

According to another exemplary embodiment of the present invention, the method further comprises the step of measuring by the sensor blood gas partial pressure values over time. Furthermore, the determined trend parameter is embodied as one or more time derivatives of a measurement curve of said blood gas partial pressure values measured over time.

In a preferred embodiment, the first time derivative and/or the second time derivative may be determined in this computer-implemented method. Such a measurement curve of said blood gas partial pressure values measured over time is shown in the non-limiting example of FIG. 2. As can be gathered from FIG. 2 and as will be explained in detail hereinafter, depending on the age of the sensor and/or depending on the age of the membrane on the sensor (i.e., the time the membrane is used with the sensor), the steepness of the measurement curve when the sensor is exposed to a pressure change gets lower and lower with increasing time, see the exemplary measurements shown in FIG. 2 from day 10 to day 50. Therefore, the first time derivative of this measurement curve can be used by the presented method to detect that a re-membraning, replacement or maintenance of the sensor is needed. For example, if the first time derivative is not steep enough/too low in a particular time period of said measurement curve, for example between 50 and 300 s, the method may detect that a re-membraning is needed. In other words, in this embodiment, the trend parameter is a time derivative of the measured partial blood gas pressure over time. A decrease in the slope of the curve shown in for example FIG. 2 indicates a bad or old sensor. In other words, the method may carry out a so called “slope test”. This is then used by the presented method for the decision making of the re-membraning.

According to another exemplary embodiment of the present invention, the response time parameter determined in step S1 are values of the response time over a period of time, preferably at least a plurality of days. Furthermore, the determined values of the response time describe a response time measurement curve of values of the response time of the sensor over said period of time. Moreover, the determined trend parameter is a time derivative of the response time measurement curve.

In other words, in this embodiment, the trend parameter is a time derivative of a measurement curve as the one shown in FIG. 3. The inventors of the present invention have identified that a strong increase in the slope, i.e., the first derivative, of the shown curve indicates a phase change of the membrane. In the non-limiting example shown in FIG. 3, the strong increase of the first time derivative/slope of the curve shown in FIG. 3 occurs at around 36 days. Thus, if the absolute value of the slope/first time derivative increases above a particular threshold value, the presented method may then determine that the partial pressure measuring capability of the sensor is not sufficient any more. Said particular threshold value of the first time derivative is thus used in this embodiment as a trend threshold. Moreover, in another example, one may also use a value of the second time derivative as a trend threshold and base the determination of the sensor capability on the comparison of the value of the second time derivative with said trend threshold. As is clear to the skilled reader, in this embodiment the method compares the determined trend parameter, i.e., the first or second time derivative, with a trend threshold regarding the time development of the response time of the transcutaneous sensor, i.e., a set threshold value of the first or second time derivative, as described herein as step S3b.

Therefore, with the presented method the re-membraning interval can be extended beyond the fixed time period used in the prior art. This improves one of the biggest disadvantages for customers using transcutaneous monitoring.

According to another exemplary embodiment of the present invention, the method comprises the step of determining whether the sensor at a start of the monitoring method is inside a calibration chamber, preferably a calibration chamber of a transcutaneous blood gas monitor.

For particular applications it may be beneficial to know whether the sensor to be monitored is currently inside a calibration chamber. In a preferred embodiment, the angle of the calibration chamber door may be used to indicate whether the sensor is inside the chamber. Since the door in this embodiment is a little more open to accommodate the sensor, the angle of the calibration chamber door may be used for such an indication.

According to another exemplary embodiment of the present invention, the method comprises the step of ensuring that the sensor is exposed to a minimum amount of gas pressure change during the method of monitoring.

In particular embodiments, this can be ensured by using a calibration chamber into which calibration gas is flushed by the device carrying out the monitoring method. For example, the transcutaneous blood gas monitor described herein may cause that calibration gas is delivered from a calibration gas reservoir into the calibration chamber, see e.g., FIG. 5. In a particular, non-limiting embodiment, there may be an outlet tube between the calibration chamber and the ambient air. Therefore, when the flow from the calibration gas reservoir is closed, the air inside the calibration chamber will slowly be exchanged with atmospheric air by simple diffusion. The inventors of the present invention have determined this exchange rate. Preferably, the monitoring method suggested herein may normally only be demanded after more than 30 minutes after the previous successful monitoring, and hence the change in gas pressure should normally be sufficient to determine the response time. However, there may be some special cases/occurrences where the scenario previously described holds not true, for instance if the sensor temperature is changed after less than 30 minutes after monitoring/calibration, for example.

Moreover, the step of ensuring that the sensor is exposed to a minimum amount of gas pressure change during the method of monitoring can also be implemented by looking at the monitoring curve when the monitoring is finished. One takes the difference between the initial gas pressure value, which should be the minimum, and the final gas pressure value, which should be the maximum, and checks if that is at least a particular desired amount of gas pressure change.

As was described hereinbefore, the method of monitoring suggested herein may be carried out by a transcutaneous monitor, which comprises the transcutaneous sensor, a calibration chamber and a calibration gas reservoir. In this embodiment, the method comprises the step of flushing calibration gas of the calibration gas reservoir into the calibration chamber, in which the transcutaneous sensor is placed for monitoring of the sensor.

According to another exemplary embodiment of the present invention, the step S1 of determining the response time parameter comprises providing data of the sensor and/or data about the sensor to a predictive maintenance module as input data. Such data may be considered as monitoring data. Such a predictive maintenance module may be embodied as an artificial intelligence module and/or a module, which was trained by machine learning. The method of this embodiment may further comprise predicting, by the predictive maintenance module, the response time of the transcutaneous sensor. Furthermore, in step S2, the predicted response time of the transcutaneous sensor is used for the determination whether a partial pressure measuring capability of the sensor is sufficient, particularly whether a re-membraning of the transcutaneous sensor or a replacement or maintenance of the sensor is necessary or needed.

Such a predictive maintenance module may thus predict values of the measurement curves shown for example in FIGS. 2 and 3. In other words, instead of measuring a partial blood gas pressure as carried out in FIG. 2, and/or instead of measuring/determining the response time over membrane age, as carried out in FIG. 3, such measurement curves may be predicted by the predictive maintenance module. These predicted response time values of the transcutaneous sensor may then be used for the decision making in step S2 whether the partial pressure measuring capability of the sensor is still sufficient or will be sufficient at a future point in time. Preferably, in step S2 it is determined, based on the determined response time parameter, whether a partial pressure measuring capability of the sensor is sufficient thereby determining whether the determined response time parameter fulfils or meets a sufficiency criterion, i.e., an acceptance criterion.

In general, an artificial intelligence module is an entity that processes one or more inputs into one or more outputs by means of an internal processing chain that typically has a set of free parameters. The internal processing chain may be organized in interconnected layers that are traversed consecutively when proceeding from the input to the output. Many artificial intelligence modules are organized to process an input having a high dimensionality into an output of a much lower dimensionality. A common job for an artificial intelligence module is to classify data into one or more categories. Such a module is termed “intelligent” because it is capable of being “trained.” The module may be trained using records of training data. A record of training data comprises training input data and corresponding training output data. The training output data of a record of training data is the result that is expected to be produced by the module when being given the training input data of the same record of training data as input. The deviation between this expected result and the actual result produced by the module is observed and rated by means of a “loss function”. This loss function is used as a feedback for adjusting the parameters of the internal processing chain of the module. For example, the parameters may be adjusted with the optimization goal of minimizing the values of the loss function that result when all training input data is fed into the module and the outcome is compared with the corresponding training output data.

The result of this training is that given a relatively small number of records of training data as “ground truth”, the module is enabled to perform its job well for a number of records of input data that are higher by many orders of magnitude. In the context of this embodiment, neural networks, convolutional neural networks and generative adversarial networks may be used.

According to another exemplary embodiment of the present invention, the method is applied to a transcutaneous electrochemical sensor for measuring partial pressure of one or more blood gases of a patient.

While it has been described hereinbefore that also optical sensors may be monitored with the present invention, this particular embodiment relates to electrochemical sensors. In particular, electrochemical sensors are often used for measuring the partial pressure of CO₂ of patients. Effects of drying of the membrane, as detailed hereinbefore, can be avoided or reduced for electrochemical sensors. The re-membraning interval can be extended beyond the fixed time period used by the prior art. Moreover, with the presented method, a electrochemical sensor can be detected that for some reason has dried out before the fixed time period used in the prior art has elapsed.

According to another aspect of the present invention, a program which, when running on a computer or when loaded onto a computer, causes the computer to perform the method steps of the method as disclosed herein.

The program may be part of a computer program, but it can also be an entire program by itself. For example, the program may be used to update an already existing computer program to get to the present invention.

According to another aspect of the present invention, a non-transitory program storage medium storing such a program is presented.

The computer readable medium may be seen as a storage medium, such as for example, a data storage device, a USB stick, a CD, a DVD, a hard disk, or any other medium on which a program as described above can be stored.

According to another exemplary embodiment of the present invention, a transcutaneous blood gas monitor for transcutaneously monitoring one or more blood gases of a patient is presented. The monitor comprises a transcutaneous, preferably electrochemical, sensor configured for measuring partial pressure of one or more blood gases of the patient. The monitor further comprises a controlling circuitry, which is configured, during monitoring of the transcutaneous sensor, for determining a response time parameter of the transcutaneous sensor. Moreover, the controlling circuitry is configured for determining, based on the determined response time parameter, whether a partial pressure measuring capability of the sensor is sufficient.

A particular non-limiting example of such a blood gas monitor is depicted in FIG. 5 and will be explained in more detail hereinafter. Preferably, the controlling circuitry is configured for determining, based on the determined response time parameter, whether a partial pressure measuring capability of the sensor is sufficient thereby being configured for determining whether the determined response time parameter fulfils or meets a sufficiency criterion, i.e., an acceptance criterion.

According to another exemplary embodiment of the present invention, the transcutaneous blood gas monitor comprises a calibration chamber and a calibration gas reservoir. The transcutaneous blood gas monitor is configured for flushing calibration gas of the calibration gas reservoir into the calibration chamber for calibrating the transcutaneous sensor.

In a particular embodiment, the transcutaneous blood gas monitor is configured for ensuring that the sensor is exposed to a minimum amount of gas pressure change during the method of monitoring.

According to another aspect of the present invention, the use of a result of a determination of a response time parameter of a transcutaneous sensor, which is configured for measuring partial pressure of one or more blood gases of a patient, for monitoring of the sensor, is presented. In particular, this may be used for determining a need for re-membraning of the transcutaneous sensor or for determining a need for replacing the sensor due to deterioration. Preferably, the results are used in or by a transcutaneous blood gas monitor, computer-implemented method, by a program and/or by a computer. Note that the result of the determination of the response time parameter of a transcutaneous sensor are preferably data that can be used as the input for such a transcutaneous blood gas monitor, computer-implemented method, a program and/or by a computer. Thus, the disclosed used is not to be understood as a mental act but as the technical use of the data generated by said determination.

According to an exemplary embodiment of the present invention, the transcutaneous blood gas monitor is configured for regularly carrying out the method as presented herein, for example every 24 hours, every 12 hours, every 8 hours, or the like.

### BRIEF DESCRIPTION OF THE DRAWINGS

In the following, the invention is described with reference to the appended figures which give background explanations and represent specific embodiments of the invention. The scope of the invention is, however, not limited to the specific features disclosed in the context of the figures.

FIG. 1 schematically shows a flow diagram of a computer-implemented method of monitoring a transcutaneous sensor according to an embodiment of the present invention.

FIG. 2 shows four measurement curves of the partial CO₂ pressure over time which can be used in one or more embodiments of the present invention.

FIG. 3 schematically shows a measurement curve of a response time of a transcutaneous sensor over the membrane age which can be used in one or more embodiments of the present invention.

FIG. 4 schematically shows a theoretical response curve of a transcutaneous sensor for measuring partial pressure of one or more blood gases of a patient when exposed to a pressure change that can be used in one or more embodiments of the present invention.

FIG. 5 schematically shows a transcutaneous blood gas monitor for transcutaneously monitoring one or more blood gases of a patient according to an embodiment of the present invention.

FIG. 6 schematically shows a flow diagram of a computer-implemented method of monitoring a transcutaneous sensor according to an embodiment of the present invention.

### DESCRIPTION OF EMBODIMENTS

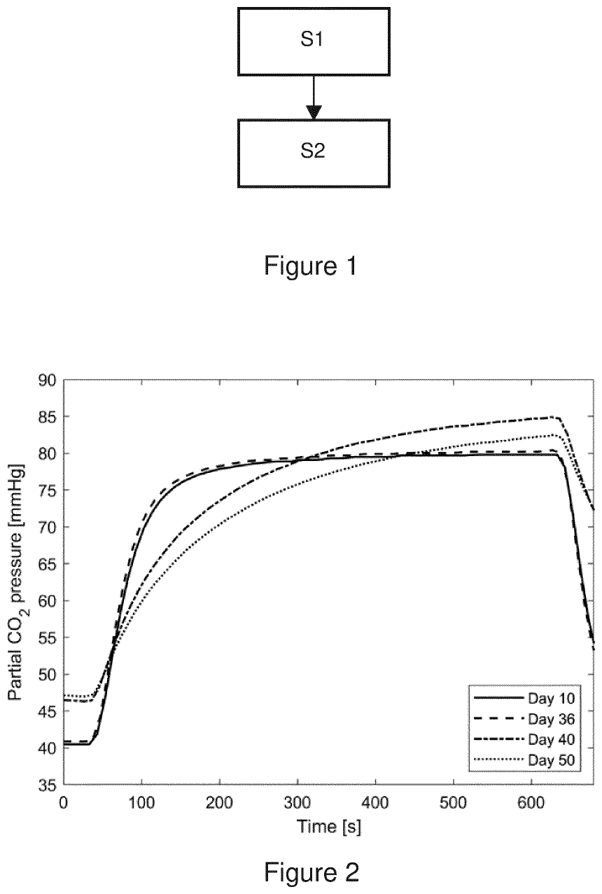

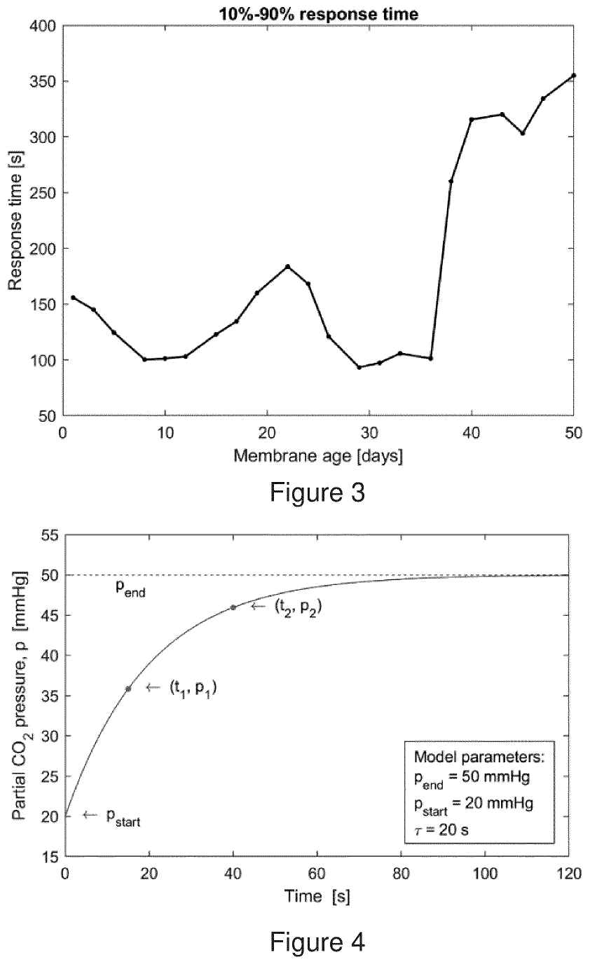

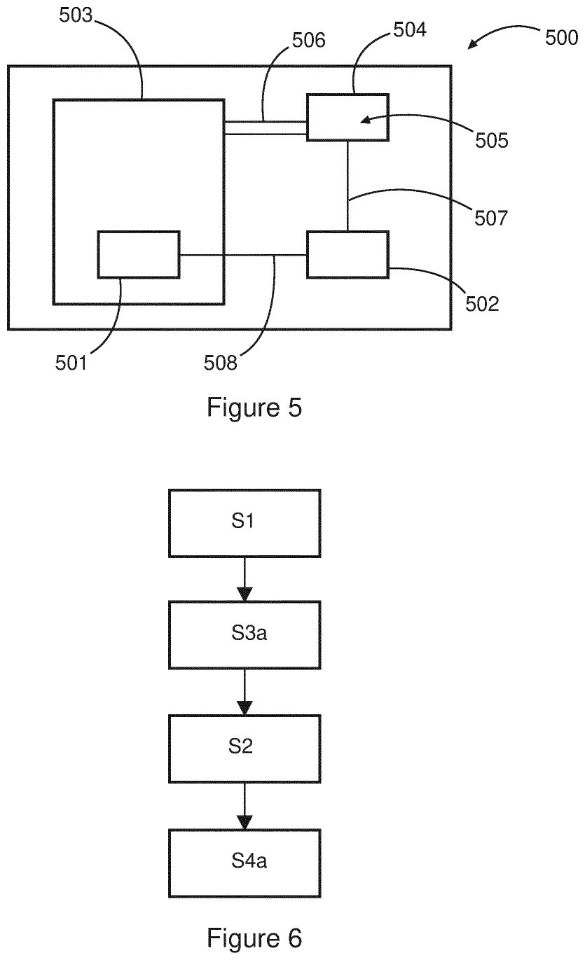

FIG. 1 schematically shows a flow diagram of a computer-implemented method of monitoring a transcutaneous sensor configured for measuring a partial pressure of one or more blood gases of a patient. Preferred blood gases are oxygen and carbon dioxide, however, also other blood gases may be measured. As has been explained hereinbefore, the presented method shown in FIG. 1 can be applied to transcutaneous electrochemical sensors, but the method is also applicable to transcutaneous sensors measuring partial pressure of blood gases of patients using optical technologies. The method presented in FIG. 1 comprises step S1 in which a response time parameter of the transcutaneous sensor is determined. Said response time parameter may be one or more absolute values of the response time of the sensor, but may also be a trend parameter like for example a first or a second derivative of, for example, a measurement curve shown in FIGS. 2 and 3. Furthermore, in a second step S2, it is determined by the method based on the response time parameter that was determined before, whether a partial pressure measuring capability of the sensor is sufficient, adequate or acceptable. As will be appreciated by the skilled reader, in the context of the present invention, the term “sufficient” shall be used synonymously with the term “adequate” and “acceptable”. Different possibilities like e.g., the comparison of a measured response time with a response time threshold are disclosed for determining whether the capability of the sensor is sufficient or not. Thus, in general, the method determines in step S2 whether the determined response time parameter fulfils or meets a sufficiency criterion, i.e., an acceptance criterion. This sufficiency criterion, i.e., an acceptance criterion, may preferably be predefined. Thus, as is understood by the skilled reader, in step S2 it is determined, based on the determined response time parameter, whether the partial pressure measuring capability of the sensor is considered—by the computer implemented method—as acceptable/accepted. Therefore, step S2 is to be understood as determining, based on the determined response time parameter, whether a partial pressure measuring capability of the sensor is sufficient thereby determining whether the determined response time parameter fulfils or meets a sufficiency criterion, i.e., an acceptance criterion. The method shown in FIG. 1 may be carried out by e.g., a controlling circuitry, a processor or a computer. Such a controlling circuitry, processor or computer may be comprised by a transcutaneous blood gas monitor 500 shown in the non-limiting example of FIG. 5.

FIG. 2 shows the measurement curve of a transcutaneous sensor for measuring partial pressure of carbon dioxide (CO₂) over time. As can be gathered from FIG. 2, four different measurements were carried out at day 10, 36, 40 and 50. The days indicate the age of the membrane used on the sensor. At each measurement the sensor was exposed to an external gas pressure. As can be gathered from FIG. 2, the slope of the respective curve significantly differs between the sensor measurements at use on day 10 compared to the sensor measured at use on day 50. Therefore, in an exemplary embodiment, the steepness of this measurement curve, i.e., the first time derivative of the mathematical function describing the depicted curve, may be used as a criterion for the determination in step S2 of FIG. 1, whether the partial pressure measuring capability of the sensor is sufficient. A decrease in the slope of the curve shown in FIG. 2 indicates a bad or old sensor. This is then used by the presented method for the decision making, e.g., for re-membraning. In other words, according to particular embodiments of the present invention, one may use for the trend parameter described herein the first and/or the second time derivative of the measurement curve as shown in FIG. 2. Moreover, the trend threshold used in step S3b in preferred embodiments is a set threshold value of the first and/or second time derivative of said measurement curve shown in FIG. 2.

FIG. 3 shows the so called the 10% to 90% response time calculated for a plurality of days. The calculation of said response time will be explained in detail in the context of FIG. 4. As can be gathered from FIG. 3, a strong increase in the slope, i.e., the first derivative of the shown curve, indicates a phase change of the properties of the membrane. In the example of FIG. 3, the strong increase occurs at around 36 day of membrane age. Therefore, the method presented herein can detect the need for a re-membraning which only occurs after 36 days, and not after 28 days, which is currently used in the prior art as a fixed interval for re-membraning. For this detection an embodiment of the presented method determines as a response time parameter in step S1 one or more values of the first time derivative and/or a second time derivative as a trend parameter of the response time curve over time shown in FIG. 3. This determined first time derivative and/or a second time derivative is then compared with a trend threshold as described herein as step S3b. Said trend threshold may be a, preferably predetermined, value of the first and/or second time derivative. Thus, if e.g., the absolute value of the slope/first time derivative increases above a particular threshold value, as is the case for the curve of FIG. 3 at around 36 days, the presented method may then determine that the partial pressure measuring capability of the sensor is not sufficient anymore.

This example makes clear that with the method suggested herein, the re-membraning interval can be extended beyond the known prior art period. Moreover, the presented method can detect a sensor that for some reason has dried out before the prior art time period has elapsed. Moreover, the method presented herein can detect the sensor where the performance has decreased over time and due to use, such that the response time is too high. In such a case, the method described in the context of FIG. 3 can generate a control signal that a replacement or maintenance of the transcutaneous sensor because of deterioration is to be initiated.

FIG. 4 shows the theoretical response curve of a transcutaneous sensor to a change in gas pressure using the model parameters listed in FIG. 4. The two points used for calculating the response time in the two-point estimation are also shown in FIG. 4. The method presented herein may make use of the fact that transcutaneous sensors have a bi-exponential or exponential response to a sudden change in gas pressure p(t)=(p_{end}−p(0)) (1−e^{(+/T)})+p(0), and the time constant (τ) in the exponential response can be determined by:

$$τ = \frac{{t}_{2} - {t}_{1}}{ln ( \frac{{p}_{end} - p ( {t}_{1} )}{{p}_{end} - p ( {t}_{2} )} )} , {t}_{2} > {t}_{1}$$

Where p_{end} is the steady state value of the gas pressure in the exponential function, p(t₁) and p(t₂) are the gas pressures of the exponential function at time points t₁ and t₂ as illustrated in FIG. 4. Further, the skilled reader is aware that this time constant τ can be converted by calculation into the so called 10% to 90% response time. Note that in the context of the present invention and if not stated differently, the response time shall be understood as the so called 10% to 90% response time, i.e., the time which is needed by the sensor to detect a change from a first partial gas pressure to a second partial gas pressure, where 10% and 90% refer to the time difference between detecting 10% and 90% of the difference between the first and second partial gas pressures. This can also be related to the time constant T described herein, see e.g., the description of FIG. 4. The response time is not necessarily the 10% to 90% response time, but could in other embodiments be, e.g., the 20% to 80% response time, the 5% to 95% response time or any other percentages chosen on the response curve.

FIG. 5 schematically shows a transcutaneous blood gas monitor 500 for transcutaneously monitoring one or more blood gases of a patient. The monitor 500 comprises a transcutaneous sensor 501 which is configured for measuring a partial pressure of one or more blood gases of the patient. The monitor 500 also comprises a controlling circuitry 502 which is configured during monitoring of the sensor 501 for determining a response time parameter of the transcutaneous sensor. The controlling circuitry 502 is also configured for determining based on the determined response time parameter whether a partial pressure measuring capability of the sensor 501 is sufficient, adequate or acceptable. The monitor 500 comprises a calibration chamber 503 into which calibration gas can be flushed from the calibration gas reservoir 504. The calibration gas 505 when flushed into the calibration chamber 503 through duct 506 ensures that the sensor 501 is exposed to a minimum amount of gas pressure change during the method of monitoring carried out by the monitor 500. The controlling circuitry 502 is connected with the calibration gas reservoir 504 via electrical connections 507 to initiate the flushing. Moreover, controlling circuitry 502 is connected to the sensor 501 via connections 508 such that measurement values generated by the sensor 501 can be transmitted from the sensor 501 to the controlling circuitry 502.

FIG. 6 schematically shows a computer-implemented method of monitoring a transcutaneous sensor according to another embodiment of the present invention. In step S1, a response time parameter of the transcutaneous sensor is determined. In the embodiment of FIG. 6, a value, preferably an absolute value, of the 10% to 90% response time is determined. Moreover, in step S3a, the determined value of the 10% to 90% response time is compared with a response time threshold. As has been explained in detail hereinbefore, the threshold can be predefined like, for example, a certain fixed amount of time, e.g., 50 s. However, the threshold may also be embodied as a value that is calculated based on the response time measured when the sensor has been re-membraned. For example, the threshold may be set to 1.5 times the value of the response time measured after the re-membraning of said individual sensor. Other possibilities are disclosed hereinbefore. Moreover, in the method of FIG. 6, the determined value of the response time is compared with a response time threshold in step S3a. Furthermore, based on said comparison of step S3a, it is determined in step S2 whether a partial pressure measuring capability of the sensor is sufficient. In other words, this determination is based on the comparison of/in step S3a of the value of the response with the threshold. In particular, depending on the comparison of the determined response time with the threshold in step S3a, it can be decided that a re-membraning is necessary. For example, if the response time exceeds the threshold, it is determined by the method that re-membraning should be initiated. Therefore, a control signal for causing a re-membraning is generated in step S4a.

The described embodiments similarly pertain to the computer-implemented method of monitoring a transcutaneous sensor configured for measuring partial pressure of one or more blood gases of a patient, to the computer program, the non-transitory program storage medium storing such a program, the transcutaneous blood gas monitor for transcutaneously monitoring one or more blood gases of a patient, and to the use of a result of a determination of a response time parameter of a transcutaneous sensor. Synergetic effects may arise from different combinations of the embodiments although they might not be described in detail.

Further on, it shall be noted that all embodiments of the present invention concerning a method, might be carried out with the order of the steps as described, nevertheless this has not to be the only and essential order of the steps of the method. The herein presented methods can be carried out with another order of the disclosed steps without departing from the respective method embodiment, unless explicitly mentioned to the contrary hereinafter.

Where an indefinite or definite article is used when referring to a singular noun, e.g. “a”, “an” or “the”, this includes a plurality of that noun unless something else is specifically stated. The terms “about” or “approximately” in the context of the present invention denote an interval of accuracy that the person skilled in the art will understand to still ensure the technical effect of the feature in question. The term typically indicates deviation from the indicated numerical value of ±20%, preferably ±15%, more preferably ±10%, and even more preferably ±5%.

Other variations to the disclosed embodiments can be understood and effected by those skilled in the art in practicing the claimed invention, from the study of the drawings, the disclosure, and the appended claims. In the claims the word “comprising” does not exclude other elements or steps and the indefinite article “a” or “an” does not exclude a plurality. A single processor or other unit may fulfil the functions of several items or steps recited in the claims. The mere fact that certain measures are recited in mutually different dependent claims does not indicate that a combination of these measures cannot be used to advantage. A computer program may be stored/distributed on a suitable medium such as an optical storage medium or a solid-state medium supplied together with or as part of other hardware, but may also be distributed in other forms, such as via the Internet or other wired or wireless telecommunication systems. Any reference signs in the claims should not be construed as limiting the scope of the claims.

1. A computer-implemented method of monitoring a transcutaneous sensor configured for measuring partial pressure of one or more blood gases of a patient, the method comprising the steps:
determining a response time parameter of the transcutaneous sensor (step S1), and
determining, based on the determined response time parameter, whether a partial pressure measuring capability of the sensor is sufficient (step S2).

2. The method according to claim 1, wherein the response time parameter is indicative of the response time of the sensor in measuring a partial pressure of the one or more blood gases of the patient.

3. The method according to claim 1, wherein the step S2 of determining whether the partial pressure measuring capability of the sensor is sufficient comprises:
determining, based on the determined response time parameter, whether a re-membraning of the transcutaneous sensor is to be initiated.

4. The method according to claim 1, wherein the step S2 of determining whether the partial pressure measuring capability of the sensor is sufficient comprises:
determining, based on the determined response time parameter, whether a replacement or maintenance of the transcutaneous sensor due to deterioration is to be initiated.

5. The method according to claim 1, wherein the response time parameter determined in step S1 is a value of the response time; and the method further comprising the step:
comparing the determined value of the response time with a response time threshold (step S3a).

6. The method according to claim 5, the method further comprising the step:
generating a control signal configured for causing a re-membraning of the sensor or configured for causing a replacement or maintenance of the sensor if the determined value of the response time exceeds the response time threshold (step S4a).

7. The method according to claim 1, wherein the response time parameter determined in step S1 is a trend parameter indicative of a time development of the response time of the transcutaneous sensor; and the method further comprising the step:
comparing the determined trend parameter with a trend threshold regarding the time development of the response time of the transcutaneous sensor (S3b).

8. The method according to claim 7, the method further comprising:
generating a control signal configured for causing a re-membraning of the sensor or configured for causing a replacement or maintenance of the sensor if the determined trend parameter deviates from the trend by at least a predetermined minimum amount (S4b).

9. The method according to claim 7, the method further comprising the step:
measuring, by the sensor, blood gas partial pressure values over time, and
wherein the determined trend parameter is one or more time derivatives of a measurement curve of said blood gas partial pressure values measured over time.

10. The method according to claim 7, wherein the response time parameter determined in step S1 comprises values of the response time over a period of time,
wherein the determined values of the response time describe a response time measurement curve of values of the response time of the sensor over said period of time, and
wherein the determined trend parameter is a time derivative of the response time measurement curve.

11. The method according to claim 1, wherein the step S1 of determining the response time parameter comprises:
providing one or both of data of the sensor or data about the sensor to a predictive maintenance module as input data,
predicting by the predictive maintenance module the response time of the transcutaneous sensor; and
wherein in step S2 the predicted response time of the transcutaneous sensor is used for the determination of whether a partial pressure measuring capability of the sensor is sufficient.

12. The method according to claim 1, wherein the method is applied to a transcutaneous electrochemical sensor for measuring partial pressure of one or more blood gases of a patient.

13. The method according to claim 1, wherein the sensor is exposed to a pressure change from a first partial gas pressure to a second partial gas pressure during the method of monitoring, and
wherein one or both of said first partial gas pressure or said second partial gas pressure is/are known.

14. A program which, when running on a computer or when loaded onto a computer, causes the computer to perform the method steps of the method according to claim 1.

15. A non-transitory program storage medium storing a program according to claim 14.

16. A transcutaneous blood gas monitor (500) for transcutaneously monitoring one or more blood gases of a patient, the monitor comprising:
a transcutaneous sensor (501) configured for measuring partial pressure of one or more blood gases of the patient,
a controlling circuitry (502), which is configured, during monitoring of the transcutaneous sensor, for:
determining a response time parameter of the transcutaneous sensor,
the controlling circuitry being further configured for:
determining, based on the determined response time parameter, whether a partial pressure measuring capability of the sensor is sufficient.

17. The transcutaneous blood gas monitor according to claim 16, the monitor further comprising:
a calibration chamber (503),
a calibration gas reservoir (504) comprising calibration gas (505), and
wherein the transcutaneous blood gas monitor is configured for flushing calibration gas of the calibration gas reservoir into the calibration chamber for calibrating the transcutaneous sensor.

18. (canceled)

19. The method according to claim 10, the period of time comprising at least a plurality of days.

20. The method according to claim 11, the determination of whether a partial pressure measuring capability of the sensor is sufficient comprising a determination of whether a re-membraning of the transcutaneous sensor or a replacement or maintenance of the sensor is necessary.

21. The transcutaneous blood gas monitor according to claim 16, sensor comprising an electrochemical sensor.
